(19) United States
(12) Patent Application Publication
(10) Pub. No.: US 2017/0042486 A1
(43) Pub. Date: Feb. 16, 2017

(54) WEARABLE DEVICE CAPABLE OF PREVENTING HEATSTROKE

(71) Applicant: COMPAL ELECTRONICS, INC., Taipei City (TW)

(72) Inventor: Lien Chih Tsai, Taipei City (TW)

(21) Appl. No.: 14/887,587

(22) Filed: Oct. 20, 2015

(30) Foreign Application Priority Data
Aug. 13, 2015 (TW) 104126450

(51) Int. Cl. A61B 5/00; A61B 5/01

(52) U.S. Cl. CPC A61B 5/746; A61B 5/01; A61B 5/7282; A61B 5/742; A61B 5/7455; A61B 5/7405; A61B 5/747; A61B 5/6898; A61B 5/6833; A61B 2560/0252; A61B 2562/029; A61B 2562/0271; A61B 2562/18; A61B 2560/0242

(57) ABSTRACT

A wearable device is disclosed. The wearable device comprises an ambient temperature sensor, an ambient humidity sensor, an ultraviolet detector, a skin temperature sensor, an alarm module and a process unit. The ambient temperature sensor, the ambient humidity sensor and the ambient humidity sensor detect an ambient temperature, an ambient humidity and an ultraviolet index nearby the wearable device, respectively. The skin temperature sensor contacts with the user's skin and detects a skin temperature of the user. The process unit stores at least one preset heatstroke condition and determines whether heatstroke occurs based on the detected results of the ambient temperature, the ambient humidity, the ultraviolet index and/or the skin temperature and the at least one preset heatstroke condition. The alarm module is enabled by the process unit to generate at least one alarm signal when the process unit determines that the at least one preset heatstroke is met.

### CROSS-REFERENCE TO RELATED APPLICATION

This application claims priority from Taiwan Patent Application No. 104126450, filed on Aug. 13, 2015, the entire contents of which are incorporated herein by reference for all purposes.

### FIELD OF THE INVENTION

The present invention relates to a wearable device, and more particularly to a wearable device capable of preventing heatstroke.

### BACKGROUND OF THE INVENTION

There are a lot of sports enthusiasts around the world. In sweltering hot weather, such as summer time or midday, when people take exercises and expose their body under the direct sunshine for a long time, it is easy to cause heatstroke because of the negligence of high temperature and humidity.

At present, several wearable devices are disclosed. Taiwan patent number M487052 discloses a physiology measuring device for recording the physiological status of a user and facilitating the user to adjust the body to exercise. Taiwan patent number M487716 discloses a sensing type touch device applied to wearable device having physiological signal measurement function, wherein the wearable device could provide the user with physiological signals for acquainting his physiological status, such as the rate of heartbeat. In addition, Taiwan patent number M489621 also discloses a physiological measuring device for providing the user with physiological signals, such as the rate of heartbeat. Furthermore, US laid-open patent application publication No. US20110092790A1 discloses a biosensor, which can obtain physiological data from an individual, and may collect electrodermal activity, skin temperature, and other information. Additionally, US laid-open patent application publication No. US20140200637A1 discloses a personal thermal regulating device for remotely inducing warming of the peripheral anatomy of a user according to the body temperature of the user. Accordingly, the above-mentioned devices could detect and display several specific physiological statuses, but fail to disclose wearable devices capable detecting and providing precautionary measures for preventing heatstroke.

Recently, although some wearable devices capable of providing reminder mechanism for preventing heatstroke are developed, the wearable devices determine whether the heatstroke occurs and generate alarm signals to the user accordingly by detecting the ambient temperature and the ambient humidity merely.

However, the heatstroke might be caused by several factors, not only the ambient temperature and the ambient humidity, but also ultraviolet index and the body temperature. It is obvious that the above-mentioned devices fail to provide elements in determining whether the heatstroke occurs according to the ambient temperature, the ambient humidity, the ultraviolet index and the body temperature comprehensively. Consequently, the devices can't provide correct information and accurately determine the heatstroke due to the lack of detecting factors.

Therefore, there is a need of providing a wearable device capable of preventing heatstroke in order to overcome the above drawbacks.

### SUMMARY OF THE INVENTION

An object of the present invention provides a wearable device, which comprises an ambient temperature sensor, an ambient humidity sensor, an ultraviolet detector and a skin temperature sensor to detect an ambient temperature, an ambient humidity, an ultraviolet index and a skin temperature (i.e. the important factors causing the heatstroke), respectively, and determines whether the heatstroke might occur according to the analyzed result based on the detected factors and the preset heatstroke conditions, thereby the alarm module is instructed to issue an alarm notification to inform the user. Consequently, the user can take some precautionary measures against the heatstroke in advance accurately and immediately.

In accordance with an aspect of the present invention, there is provided a wearable device comprising an ambient temperature sensor configured to detect an ambient temperature near by the wearable device; an ambient humidity sensor configured to detect an ambient humidity nearby the wearable device; an ultraviolet detector configured to detect an ultraviolet index nearby the wearable device; a skin temperature sensor configured to detect a skin temperature of a skin of a user; an alarm module configured to selectively generate at least one alarm notification; and a process unit electrically connected with the ambient temperature sensor, the ambient humidity sensor, the ultraviolet detector, the skin temperature sensor and the alarm module, configured to store at least one preset heatstroke condition, and determine whether heatstroke occurs based on the detected results of the ambient temperature, the ambient humidity, the ultraviolet index and/or the skin temperature; wherein when the process unit determines the at least one preset heatstroke condition is met, the alarm module generates at least one alarm notification corresponding to the preset heatstroke condition.

The above contents of the present invention will become more readily apparent to those ordinarily skilled in the art after reviewing the following detailed description and accompanying drawings, in which:

### BRIEF DESCRIPTION OF THE DRAWINGS

FIG. 1 is a block diagram of a wearable device according to one preferred embodiment of the present invention;

FIG. 2 is a perspective top view of the wearable device of FIG. 1;

FIG. 3 is a perspective bottom view of the wearable device of FIG. 1;

FIG. 4 is a partially schematic cross-sectional view illustrating the wearable device of FIG. 1; and

FIG. 5 is a schematic perspective view showing a skin temperature sensor of FIG. 1 disposed on a thermal conductive substrate.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

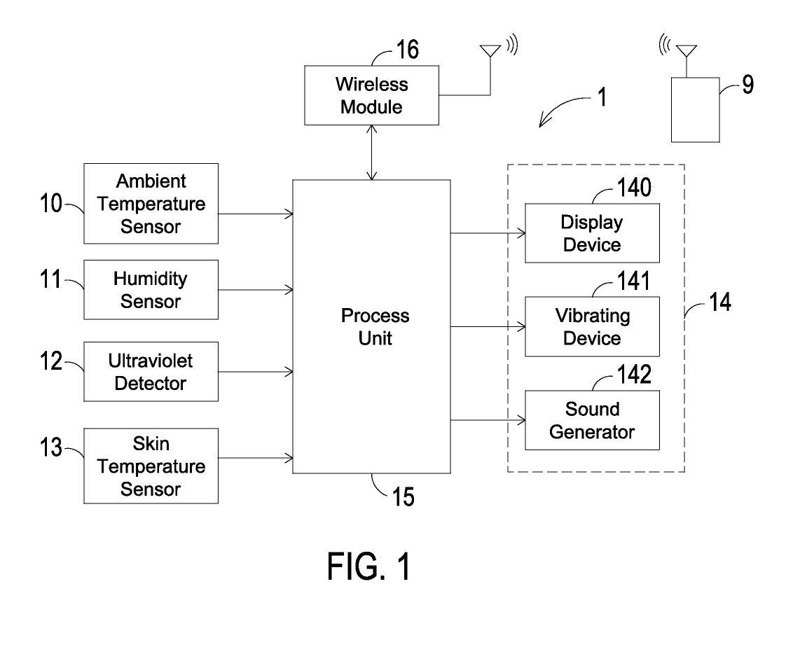

FIG. 1 is a block diagram of a wearable device according to one preferred embodiment of the present invention. As shown in FIG. 1, the wearable device 1 could be put on a user's body such as user's wrist, and comprises an ambient temperature sensor 10, an ambient humidity sensor 11, an ultraviolet detector 12, a skin temperature sensor 13, an alarm module 14, and a process unit 15. The ambient temperature sensor 10 is configured to detect an ambient temperature nearby the wearable device 1. The ambient humidity sensor 11 is configured to detect an ambient humidity nearby the wearable device 1. The ultraviolet detector 12 is configured to detect an ultraviolet index nearby the wearable device 1. Furthermore, the skin temperature sensor 13 is configured to detect a skin temperature of the skin of the user when the user wears the wearable device 1 and the skin of the user is in contact with the skin temperature sensor 13.

The alarm module 14 is electrically connected with the process unit 15 and configured to generate at least one alarm notification, wherein the at least one alarm notification includes but not limited to a warning text message, a warning vibration and/or a warning sound. The process unit 15 is but not limited to a microcontroller unit (MCU) or a central processing unit (CPU). Furthermore, the process unit 15 is electrically connected with the ambient temperature sensor 10, the ambient humidity sensor 11, the ultraviolet detector 12, the skin temperature sensor 13, and the alarm nodule 14, and stores at least one preset heatstroke condition. The process unit 15 is configured to control the operations of the ambient temperature sensor 10, the ambient humidity sensor 11, the ultraviolet detector 12, the skin temperature sensor 13, and the alarm module 14. Moreover, the process unit 15 receives the detection result from the ambient temperature sensor 10, the ambient humidity sensor 11, the ultraviolet detector 12 and/or the skin temperature sensor 13, and further determines whether the heatstroke occurs based on the detected results of the ambient temperature, the ambient humidity, the ultraviolet index and/or the skin temperature. When the detected result meets the preset heatstroke condition, the alarm module 14 is instructed by the process unit 15 to generate at least one alarm notification corresponding to the preset heatstroke condition for informing or reminding the user to take a corresponding precautionary measure, thereby preventing heatstroke.

In this embodiment, the alarm module 14 comprises a display device 140, a vibrating device 141 and a sound generator 142. The display device 140 is but not limited to a liquid crystal display (LCD) or a light emitting diode (LED) display. The display device 140 is electrically connected with the process unit 15 and controlled by the process unit 15 for displaying a warning text message. In some embodiment, the display device 140 is instructed by the process unit 15 to display the ambient temperature detected by the ambient temperature sensor 10, the ambient humidity detected by the ambient humidity sensor 11, the ultraviolet index detected by the ultraviolet detector 12, and the skin temperature detected by the skin temperature sensor 13 for allowing the user to view and inquire the detected results.

The vibrating device 141 is but not limited to a vibration motor. The vibrating device 141 is connected with the process unit 15 and controlled by the process unit 15. The vibrating device 141 is configured to generate a warning vibration.

The sound generator 142 is but not limited to a loudspeaker. The sound generator 142 is electrically connected with the process unit 15 and controlled by the process unit 15. The sound generator 142 is configured to generate a warning sound.

In an embodiment, the process unit 15 stores plural preset heatstroke conditions. The preset heatstroke conditions can be classified into at least three levels of heatstroke, which are defined and described as following. From slight injury (i.e. first level) to serious injury (i.e. third level), the preset heatstroke conditions are (1) the ambient temperature detected by the ambient temperature sensor 10 is over a preset ambient temperature, and the ambient humidity detected by the ambient humidity sensor 11 is over a preset ambient humidity (i.e. first level); (2) the ultraviolet index detected by the ultraviolet detector 12 is over a preset ultraviolet index for a first preset period of time, for example but not limited to one hour (i.e. second level); and (3) the skin temperature detected by the skin temperature sensor 13 is over a preset skin temperature (i.e. third level). When the detected results meet the preset heatstroke conditions at different levels, the alarm module 14 is instructed by the process unit 15 to generate respective alarm notifications corresponding to the preset heatstroke conditions at different levels. In an embodiment, when the processor unit 15 acquires the ambient temperature from the ambient temperature sensor 10 and the ambient humidity from the ambient humidity sensor 11 over the preset ambient temperature and the preset ambient humidity respectively, the process unit 15 determines that the user may suffer from heatstroke corresponding to the preset heatstroke condition at first level, and then the display device 140 is instructed by the process unit 15 to display a warning text message correspondingly.

Moreover, when the processor unit 15 acquires the ultraviolet index from the ultraviolet detector 12 over the preset ultraviolet index for the first preset period of time, the process unit 15 determines that the user may suffer from heatstroke corresponding to the preset heatstroke condition at second level, and then the display device 140 is instructed by the process unit 15 to display a warning text message and the vibrating device 141 is instructed by the process unit 15 to generate a warning vibration correspondingly.

Furthermore, when the processor unit acquires the skin temperature from the skin temperature 13 over the preset skin temperature, the process unit 15 determines that the user may suffer from heatstroke corresponding to the preset heatstroke condition at third level, and then the display device 140 is instructed by the process unit 15 to display a warning text message, the vibrating device 141 is instructed by the process unit 15 to generate a warning vibration, and the sound generator 142 is instructed by the process unit 15 to generate a warning sound correspondingly.

It is noted that the detected factors and parameters of the preset heatstroke conditions stored in the process unit 15 are not limited to the detecting factors and parameters of the preset heatstroke conditions described as above. Namely, in the above embodiments, the heatstroke condition at first level is determined according to whether the ambient temperature is over the preset ambient temperature, and whether the ambient humidity is over the preset ambient humidity. The preset heatstroke condition at second level is determined according to whether the ultraviolet index is over a preset ultraviolet index for the first preset period of time. The preset heatstroke condition at third level is determined according to whether the skin temperature is over a preset skin temperature. Alternatively, in some embodiments, the detected factors and parameters of the preset heatstroke conditions can be adjusted and varied according to the practical requirements, the local climates and/or self-health condition of the user, and it is not limited thereto.

According to the above descriptions, the acts of the display device 140, the vibrating device 141 and the sound generator 142 corresponding to the preset heatstroke conditions at different levels are illustrated as the following table.

| | Display | Vibrating | Sound |
|---|---|---|---|
| Status and condition | device 140 | device 141 | generator 142 |
| heatstroke occurs possibly | display | inoperative | inoperative |
| (First level) | warning text | | |
| | message | | |
| approach to the occurrence | display | generate | inoperative |
| of heatstroke | warning text | warning | |
| (Second level) | message | vibration | |
| heatstroke occurs | display | generate | generate |
| (Third level) | warning text | warning | warning |
| | message | vibration | sound |

In this embodiment, when the skin temperature detected by the skin temperature sensor 13 and acquired by the process unit 15 is over a preset skin temperature for a second period of time, the process unit 15 determines that the user may suffer from heatstroke corresponding to the preset heatstroke condition at third level, and the display device 140 is instructed by the process unit 15 to show a heatstroke emergency action in form of words. Consequently, the user can take some actions according to the information shown on the display device 140. In an embodiment, the heatstroke emergency action in form of words includes but not limited to reminding the user to drink lots of fluids to avoid dehydration, such as reminding the user to drink the oral rehydration solution (ORS) or sports drink. Alternatively, the heatstroke emergency action in form of words includes reminding the user to cool down the body temperature, such as reminding the user to take a shower first, or reminding the user to put a cold beverage can or ice pillow under his armpit.

In this embodiments, preferably, the preset ambient temperature is 30° C., the preset ambient humidity is 95%, the preset ultraviolet index preferably is 7, and the preset skin temperature is 37.5° C., but it is not limited.

In some embodiments, as shown in FIG. 1, the wearable device 1 further comprises a wireless module 16. The wireless module 16 is electrically connected with the process unit 15 and controlled by the process unit 15. The wireless module 16 is configured to communicate with a mobile phone 9 carried by the user selectively for performing a rescue action while the skin temperature detected by the skin temperature sensor 13 and acquired by the process unit 15 is over the preset skin temperature for the second period of time. In an embodiment, the rescue action performed by the mobile phone 9 includes but not limited to guiding the user to a shady place nearby via a built-in global positioning system (GPS) of the mobile phone 9, or calling a preset emergency phone number, such as number 110 or number 119, via the mobile phone 9. In this embodiment, the wireless module 16 is in communication with the mobile phone 9 by means of a wireless communication network such as Bluetooth low energy (BLE), Wi-Fi or other type wireless communication networks.

Accordingly, the wearable device 1 can monitor the important factors in relation to heatstroke by the ambient temperature sensor 10, the ambient humidity sensor 11, the ultraviolet detector 12, and the skin temperature sensor 13. Namely, the wearable device 1 can detect (1) the ambient temperature, (2) the ambient humidity, (3) the ultraviolet index and (4) the skin temperature by the ambient temperature sensor 10, the ambient humidity sensor 11, the ultraviolet detector 12 and the skin temperature sensor 13, respectively. The wearable device 1 could analyze the detected results and determine whether the body status of the user meets the preset heatstroke conditions at first level, second level or third level. Under this circumstance, the alarm module 14 is instructed by the process unit 15 to generate at least one corresponding alarm notification to inform the user for preventing heatstroke. Consequently, the wearable device 1 can provide accurate information to the user in real-time and protect the user from heatstroke effectively.

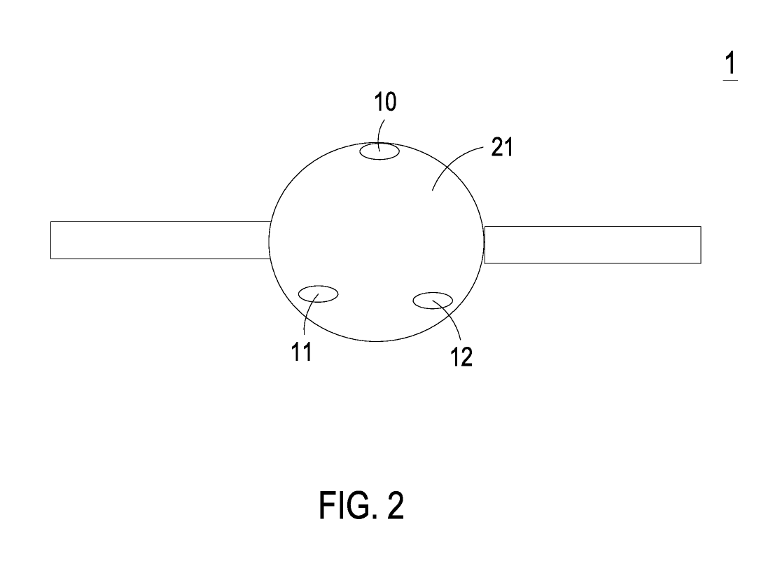

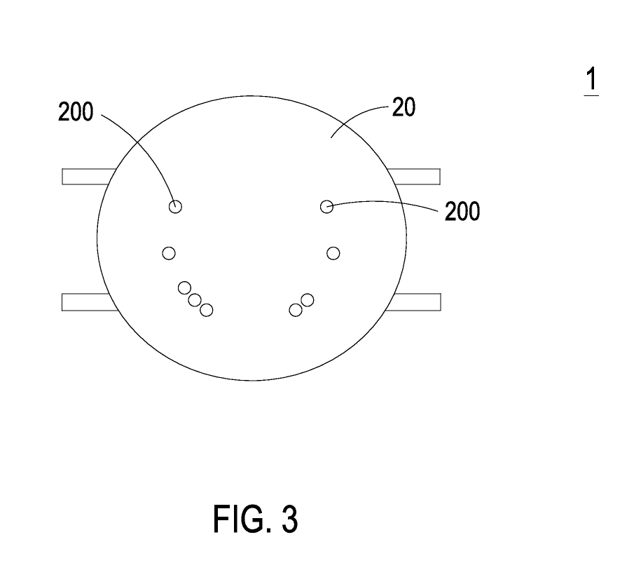

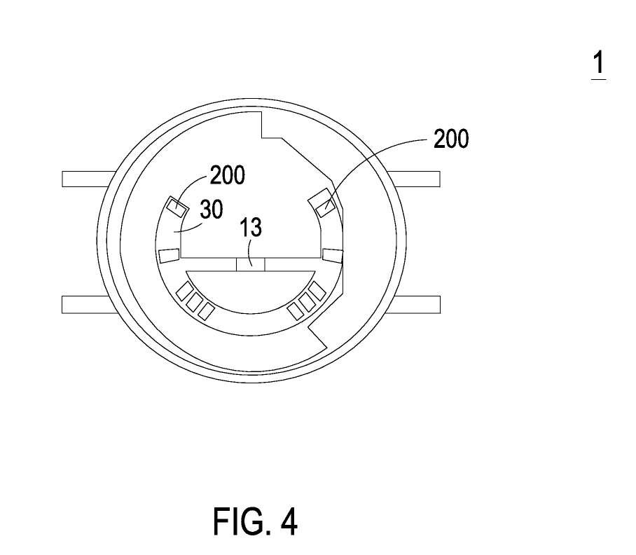

FIG. 2 is a perspective top view of the wearable device of FIG. 1, FIG. 3 is a perspective bottom view of the wearable device of FIG. 1, and FIG. 4 is a partially schematic cross-sectional view illustrating the wearable device of FIG. 1. As shown in FIGS. 2, 3 and 4, the wearable device 1 is but not limited to a smart watch, and has a first outer surface 20 and a second outer surface 21. Preferably, the first outer surface 20 is a rear surface, and the second outer surface 21 is a front surface. When the wearable device 1 is put on the wrist of the user, the first outer surface 20 is in contact with the skin of the user. The wearable device 1 includes a plurality of metallic contact pads 200 disposed on the first outer surface 20 for contacting with the skin of the user. The wearable device 1 further includes a thermal conductive substrate 30 (see FIG. 4) disposed in the interior of the wearable device 1. The metallic contact pads 200 are connected with the thermal conductive substrate 30, and the skin temperature sensor 13 is connected with the thermal conductive substrate 30. Under this circumstance, the metallic contact pads 200 can transfer the heat from the skin of the user to the skin temperature sensor 13 via the thermal conductive substrate 30, and the skin temperature sensor 13 can detect the skin temperature of the user correspondingly. Preferably, the thermal conductive substrate 30 is a printed circuit board or a ceramic substrate made from materials with thermal conductive ability. In this embodiment, the ambient temperature sensor 10, the ambient humidity sensor 11 and the ultraviolet detector 12 has respective sensing parts disposed on the second outer surface 21. The sensing parts of the ambient temperature sensor 10, the ambient humidity sensor 11 and the ultraviolet detector 12 are spaced apart.

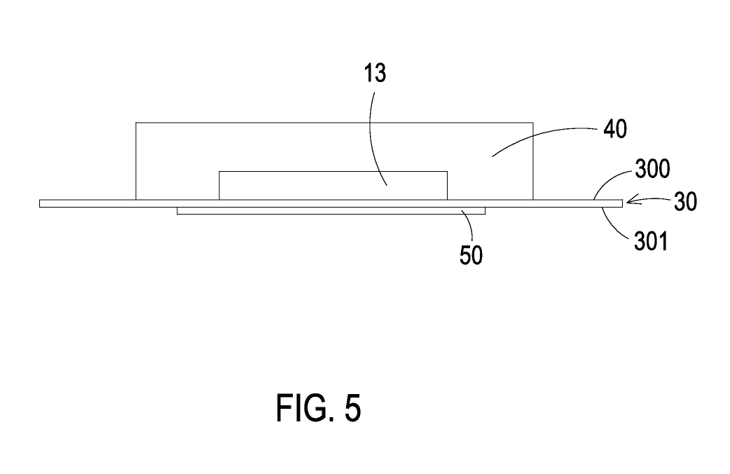

FIG. 5 is a schematic perspective view showing a skin temperature sensor of FIG. 1 disposed on a thermal conductive substrate. As shown in FIG. 5, the skin temperature sensor 13 is an integrated chip and disposed on a first mounting surface 300 of the thermal conductive substrate 30, and the thermal conductive substrate 30 is a printed circuit board with thermal conductive ability. In the embodiment, the wearable device 1 further comprises a thermal-isolating cover 40 disposed on the first mounting surface 300 of the thermal conductive substrate 30 and configured to cover the skin temperature sensor 13 for insulating the skin temperature sensor 13 and blocking thermal conduction from outside. Consequently, the issues of heat dissipation to influence the accuracy and precision of the detected results of the skin temperature sensor 13 can be prevented.

In some embodiments, the thermal conductive substrate 30 further comprises a second mounting surface 301 opposite to the first mounting surface 300. The thermal conductive substrate 30 further comprises an insulating adhesive 50 disposed on the second mounting surface 301 and corresponding to the skin temperature sensor 13. The insulating adhesive 50 is introduced to insulate the thermal conductive substrate 30 with the skin temperature sensor 13 for blocking thermal conduction from the thermal conductive substrate 30 to outside. Consequently, the issues of heat dissipation to influence the accuracy and precision of the detected results of the skin temperature sensor 13 can be prevented.

To sum up, the present invention provides a wearable device. The wearable device acquires an ambient temperature, an ambient humidity, an ultraviolet index and a skin temperature (i.e. the important factors causing the heatstroke) detected by an ambient temperature sensor, an ambient humidity sensor, an ultraviolet detector and a skin temperature sensor, respectively. The wearable device determines whether the body status of the user meets the preset heatstroke conditions at different levels based on the detected results and the preset heatstroke conditions, and further provides the user with at least one corresponding alarm notification generated by the alarm module for preventing heatstroke. Consequently, the wearable device 1 can provide accurate information in real-time and protect the user from the heatstroke effectively. Therefore, the user can take some precautionary measures against the heatstroke in advance accurately and immediately

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A wearable device, comprising:
an ambient temperature sensor configured to detect an ambient temperature near by the wearable device;
an ambient humidity sensor configured to detect an ambient humidity nearby the wearable device;
an ultraviolet detector configured to detect an ultraviolet index nearby the wearable device;
a skin temperature sensor configured to detect a skin temperature of a skin of a user;
an alarm module configured to selectively generate at least one alarm notification; and
a process unit electrically connected with the ambient temperature sensor, the ambient humidity sensor, the ultraviolet detector, the skin temperature sensor and the alarm module, configured to store at least one preset heatstroke condition, and determine whether heatstroke occurs based on the detected results of the ambient temperature, the ambient humidity, the ultraviolet index and/or the skin temperature;
wherein when the process unit determines the at least one preset heatstroke condition is met, the alarm module generates at least one alarm notification corresponding to the preset heatstroke condition.

2. The wearable device according to claim 1, wherein the alarm module further comprises:
a display device electrically connected with the process unit and controlled by the process unit for displaying a warning text message;
a vibrating device electrically connected with the process unit and controlled by the process unit for generating a warning vibration, and
a sound generator electrically connected with the process unit and controlled by the process unit for generating a warning sound,
wherein the at least one alarm notification includes at least one of the warning text message, the warning vibration and the warning sound.

3. The wearable device according to claim 2, wherein the at least one preset heatstroke condition includes a first level heatstroke condition, in which the ambient temperature detected by the ambient temperature sensor is over a preset ambient temperature and the ambient humidity detected by the ambient humidity sensor is over a preset ambient humidity.

4. The wearable device according to claim 3, wherein when the processor unit acquires the ambient temperature and the ambient humidity, and determines the ambient temperature and the ambient humidity reach the first level heatstroke condition, the processor instructs the display device to display the warning text message correspondingly.

5. The wearable device according to claim 2, wherein the at least one preset heatstroke condition includes a second level heatstroke condition, in which the ultraviolet index detected by the ultraviolet detector is over a preset ultraviolet index for a first preset period of time.

6. The wearable device according to claim 5, wherein when the processor unit acquires the ultraviolet index, and determines the ultraviolet index reaches the second level heatstroke condition, the processor instructs the display device to display the warning text message and the vibrating device to generate the warning vibration correspondingly.

7. The wearable device according to claim 2, wherein the at least one preset heatstroke condition includes a third level heatstroke condition, in which the skin temperature detected by the skin temperature sensor is over a preset skin temperature.

8. The wearable device according to claim 7, wherein when the processor unit acquires the skin temperature, and determines the skin temperature reaches the third level heatstroke condition, the processor instructs the display device to display the warning text message, the vibrating device to generate the warning vibration, and the sound generator to generate the warning sound correspondingly.

9. The wearable device according to claim 8, wherein the display device further displays a heatstroke emergency action in form of words.

10. The wearable device according to claim 8, further comprising a wireless module electrically connected with the process unit and controlled by the process unit, wherein the wireless module is configured to communicate with a mobile phone for performing a rescue action while the skin temperature reaches the third level heatstroke condition for a second period of time.

11. The wearable device according to claim 10, wherein the rescue action includes guiding the user to a shady place nearby via the mobile phone.

12. The wearable device according to claim 10, wherein the rescue action includes calling a preset emergency phone number via the mobile phone.

13. The wearable device according to claim 1, wherein the wearable device has a first outer surface and a second outer surface opposite to the first outer surface, wherein the first outer surface is in contact with the skin of the user when the wearable device is put on the user, and the ambient temperature sensor, the ambient humidity sensor and the ultraviolet detector disposed on the second outer surface.

14. The wearable device according to claim 13, further comprising:
a plurality of metallic contact pads disposed on the first outer surface for contacting with the skin of the user;
a thermal conductive substrate disposed in the interior of the wearable device and connected with the skin temperature sensor and the metallic contact pads, respectively, wherein the metallic contact pads are configured to transfer heat from the skin of the user to the skin temperature sensor via the thermal conductive substrate.

15. The wearable device according to claim 14, wherein the skin temperature sensor is an integrated chip and disposed on a first mounting surface of the thermal conductive substrate, and the thermal conductive substrate is a printed circuit board with thermal conductive ability.

16. The wearable device according to claim 15, further comprising a thermal-isolating cover disposed on the first mounting surface of the thermal conductive substrate and configured to cover the skin temperature sensor for blocking thermal conduction between the skin temperature sensor and outside.

17. The wearable device according to claim 15, further comprising a heat-insulating adhesive disposed on the second mounting surface opposite to the first mounting surface for blocking thermal conduction between the skin temperature sensor and outside.
